(19) United States
(12) Patent Application Publication
(10) Pub. No.: US 2025/0244332 A1
(43) Pub. Date: Jul. 31, 2025

(54) METHODS FOR DIAGNOSING LEPTOMININGEAL METASTASIS

(71) Applicant: H. LEE MOFFITT CANCER CENTER AND RESEARCH INSTITUTE, INC., Tampa, FL (US)

(72) Inventors: Inna SMALLEY, Tampa, FL (US); Keiran S.M. SMALLEY, Tampa, FL (US); Peter A. FORSYTH, Tampa, FL (US)

(21) Appl. No.: 18/704,790

(22) PCT Filed: Oct. 25, 2022
(86) PCT No.: PCT/US2022/047715

§ 371 Date: Apr. 25, 2024

Related U.S. Application Data
(60) Provisional application No. 63/271,578, filed on Oct. 25, 2021.

(51) Int. Cl. G01N 33/574

(52) U.S. Cl. CPC G01N 33/57488; G01N 33/574

(57) ABSTRACT

Disclosed are methods for the detection and treatment of leptomeningeal metastasis (LM).

This application claims the benefit of U.S. Application No. 63/271,578, filed on Oct. 25, 2021, which is incorporated herein by reference in its entirety.

### I. STATEMENT OF GOVERNMENT SUPPORT

This invention was made with Government support under Grant Nos CA226679, CA236034, and CA216756 awarded by the National Institutes of Health and Grant Nos. W81XWH-18-1-0268 and W81XWH-19-1-0675 awarded by the United States Department of Defense. The Government has certain rights in the invention.

### II. BACKGROUND

One of the most serious complications of advanced melanoma is the metastasis of the tumor cells into intracranial structures and the cerebrospinal fluid (CSF). The brain and spinal cord are covered with two sets of membranes, the pia mater and the arachnoid mater, that create a CSF-filled space which together are known as the leptomeninges. Leptomeningeal metastases (LM) are thought to arise from the vascular dissemination of melanoma cells, which then invade through the blood vessels of the arachnoid and choroid plexus. Other potential routes of leptomeningeal spread include the migration of cancer cells from metastases in the brain to the leptomeninges and perineural invasion, in which cancer cells migrate along the cranial or spinal nerves. The prognosis for these patients is grim and is typically associated with a mean survival of 8 to 10 weeks. Moreover, current diagnostic protocols rely heavily on a process of elimination using biopsies and microscopy. Even worse, the current diagnostics have a near 50% false negative rate leading to improper treatment and death. What are needed are new diagnostic methods that provide earlier detection LM which allows more time for therapeutic interventions to work as well as be applied to a patient in a better state of health.

### III. SUMMARY

Disclosed are methods and compositions related to the diagnosis and treatment of leptomeningeal metastasis (LM).

In one aspect, disclosed herein are methods of diagnosing leptomeningeal metastasis (LM) of a cancer (such as, for example, melanoma, breast cancer, lung cancer, leukemia, or lymphoma) in a subject comprising: a) obtaining a biological sample (such as, for example, a tissue sample, or liquid biopsy including, but not limited to blood, plasma, or cerebrospinal fluid (CSF)); and b) obtaining one or more blood gas, blood chemistry, mass spectrometry, or hematological measurements (such as, for example, a measurement of pH, PO₂, Lactate, Glucose, Sodium, Potassium, Glutamine, keto acid levels (including, but not limited to branched chain keto acids (such as, for example, 3-methyl/4-methyl-2-oxovaleric acid and 3-methyl-2-oxobutanoic acid)), amino acid levels (including, but not limited to branched chain amino acids), and/or Urea Nitrogen); wherein a significant deviation of one or more measurements relative to the same measurements from a nonmetastatic control (such as, for example, a lower pH, higher PO₂, higher lactate, lower glucose, lower potassium, lower sodium, lower glutamine, and/or higher urea nitrogen) indicates the presence of LM.

Also disclosed herein are methods of diagnosing LM of any preceding aspect, wherein the one or more blood gas, blood chemistry, or hematological measurements comprise a measurement of one or any combination of two or more of sodium (Na), potassium (K), chloride (Cl), total CO₂ (TCO₂), Anion Gap, Ionized Calcium (iCa), Lactate, 3-methyl/4-methyl-2-oxovaleric acid, 3-methyl-2-oxobutanoic acid, Glucose (Glu), Urea Nitrogen (BUN)/Urea, Creatinine, Hematocrit, Hemoglobin, pH, partial pressure of carbon dioxide (PCO₂), partial pressure of oxygen (PO₂), bicarbonate (HCO₃), Base Excess, and/or sO₂.

In one aspect, disclosed herein are methods of diagnosing LM of any preceding aspect, wherein the one or more blood gas, blood chemistry, or hematological measurements are obtained using a point-of-care body fluid analyzer (such as, for example an iSTAT). In some aspect, the one or more blood gas, blood chemistry, or hematological measurements are on a cartridge (such as, for example an iSTAT CHEM8 or CG4 cartridge) for use with a body fluid analyzer.

Also disclosed herein are methods of treating, inhibiting, reducing, decreasing, ameliorating, and/or preventing LM of a cancer (such as, for example, melanoma, breast cancer, lung cancer, leukemia, or lymphoma) in a subject comprising: a) obtaining a biological sample (such as, for example, a tissue sample, or liquid biopsy including, but not limited to blood, plasma, or cerebrospinal fluid (CSF)); b) obtaining one or more blood gas, blood chemistry, mass spectrometry, or hematological measurements (such as, for example, a measurement of pH, PO₂, Lactate, Glucose, Sodium, Potassium, Glutamine, keto acid levels (including, but not limited to branched chain keto acids (such as, for example, 3-methyl/4-methyl-2-oxovaleric acid and 3-methyl-2-oxobutanoic acid)), amino acid levels (including, but not limited to branched chain amino acids), and/or Urea Nitrogen) in the biological sample; wherein a significant deviation of one or more measurements relative to the same measurements from a nonmetastatic control (such as, for example, a lower pH, higher PO₂, higher lactate, lower glucose, lower potassium, lower sodium, lower glutamine, and/or or higher urea nitrogen) indicates the presence of LM; and c) administering to the subject anti-cancer therapy (such as, for example, radiation therapy, intrathecal chemotherapy, or immunotherapy).

In one aspect, disclosed herein are methods of treating, inhibiting, reducing, decreasing, ameliorating, and/or preventing LM of any preceding aspect, wherein the one or more blood gas, blood chemistry, or hematological measurements comprise a measurement of one or any combination of two or more of sodium (Na), potassium (K), chloride (Cl), total CO₂ (TCO₂), Anion Gap, Ionized Calcium (iCa), Lactate, Glucose (Glu), Urea Nitrogen (BUN)/Urea, 3-methyl/4-methyl-2-oxovaleric acid, 3-methyl-2-oxobutanoic acid, Creatinine, Hematocrit, Hemoglobin, pH, partial pressure of carbon dioxide (PCO₂), partial pressure of oxygen (PO₂), bicarbonate (HCO₃), Base Excess, and/or sO₂.

Also disclosed herein are methods of treating, inhibiting, reducing, decreasing, ameliorating, and/or preventing LM of any preceding aspect, wherein the one or more blood gas, blood chemistry, or hematological measurements are obtained using a point-of-care body fluid analyzer (such as, for example an iSTAT). In some aspect, the one or more blood gas, blood chemistry, or hematological measurements are on a cartridge (such as, for example an iSTAT CHEM8 or CG4 cartridge) for use with a body fluid analyzer.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows results of blood gas, blood chemistry, or hematological measurements are obtained using a point-of-care body fluid analyzer (iSTAT) and the CHEM8 and CG4 cartridges to obtain measurements of CSF in patients with and without LM.

FIG. 2 shows CSF was analyzed using iSTAT using CG4+ and Chem8 (Potassium, Sodium and Glutamine) cartridges. Patients with following tumor types included: Melanoma, Lung cancer, Breast Cancer, lymphoma, leukemia, glioma, pancreatic cancer and carcinoid tumor of stomach.

FIG. 3 shows absolute levels of BCKAs using mass spectrometry in CSF of cancer patients with no CNS involvement (control) or with LMD diagnosis from breast cancer (BC), melanoma (mel), and lymphoma (lym).

FIG. 4 shows summary of proteomics, metabolomics, and lipidomics for LMD patients.

FIG. 5 shows the difference in proteomic profiles of LMD patients relative to normal controls as measured for 184 differentially abundant proteins.

FIG. 6 shows the pathways associated with differentially abundant proteins.

FIG. 7 shows the difference in metabolomic profiles of LMD patients relative to normal controls as measured for 193 observations and 17 differentially abundant metabolites.

FIG. 8 shows the pathways associated with differentially abundant metabolites.

FIG. 9 shows the difference in lipidomic profiles of LMD patients relative to normal controls as measured for 160 observations

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Detecting LM

Up to 5% to 7% of all patients with melanoma will develop leptomeningeal melanoma metastases (LMM). The prognosis for these patients is grim and is typically associated with a mean survival of 8 to 10 weeks. A link has been suggested between the development of LMM and the presence of parenchymal brain metastases, with up to 19% of patients with melanoma having concurrent tumor in the brain and leptomeninges. There is some suggestion that the incidence of leptomeningeal metastases is rising, a likely consequence of better detection (improved imaging), the longer survival of patients with better controlled extracranial disease (such as with BRAF-MEK inhibition and immune checkpoint inhibitors), and the likelihood that the CSF space constitutes a “sanctuary” for tumor cells.

No therapies have been shown to be effective at altering the natural history of LMM in randomized clinical trials. In the majority of cases, patients are treated with off-label therapies including intrathecal (IT) chemotherapy (thiotepa, methotrexate, and liposomal cytarabine) and whole brain radiotherapy. Newly developed targeted therapies (such as BRAF inhibitors and the BRAF-MEK inhibitor combination) and immunotherapies (such as anti-CTLA-4 and anti-PD-1 antibodies) are also currently being investigated in patients withLMM(NCT 03025256). There are already some anecdotal reports of patients with LMM responding to BRAF inhibitors, the BRAF-MEK inhibitor combination, and immune checkpoint inhibitors, although the numbers of patients treated thus far remain small. There is evidence from a recent single-institutional study that systemic BRAF-MEK inhibitor therapy is associated with improved overall survival (OS) in patients with LMM compared with no targeted therapy. Although these results are clearly encouraging, the duration of responses observed is typically much shorter in duration to those seen at extracranial sites. The diagnosis and treatment for LM is complicated by the invasiveness of any diagnosis which relies on cytology and a graduated scale from not likely to positive. Given the short prognosis for positive individuals, earlier detection and a more definitive diagnosis can have profound effect on patients and could mean the earlier administration of a therapeutic regimen increasing the probability of a successful treatment. Moreover, as the present standard of care involve intrathecal administration of a therapeutic which is highly invasive. Due to the dangers of intrathecal administration, physicians are hesitant to initiate treatment unless diagnosis is definitive.

At this time, virtually nothing is known about the CSF environment of patients with LMM. Few comprehensive studies have been undertaken to define the composition of the CSF in patients with LMM, and it is unclear whether the CSF microenvironment contributes to melanoma progression or therapeutic resistance. In the current study, we have performed analysis of serial CSF specimens from patients with melanoma leptomeningeal metastases. We have used proteomics to define CSF composition from multiple patients with LMM and then performed RNA sequencing (RNA-seq) to define how CSF from patients with LMM transcriptionally reprograms melanoma cells.

In one aspect, disclosed herein are methods of diagnosing leptomeningeal metastasis (LM) of a cancer (such as, for example, melanoma, breast cancer, lung cancer, leukemia, or lymphoma) in a subject comprising: a) obtaining a biological sample (such as, for example, a tissue sample, or liquid biopsy including, but not limited to blood, plasma, or cerebrospinal fluid (CSF)); and b) obtaining one or more blood gas, blood chemistry, mass spectrometry, or hematological measurements in the biological sample (such as, for example, a measurement of pH, PO₂, Lactate, Glucose, Sodium, Potassium, Glutamine, keto acid levels (including, but not limited to branched chain keto acids (such as, for example, 3-methyl/4-methyl-2-oxovaleric acid and 3-methyl-2-oxobutanoic acid)), amino acid levels (including, but not limited to branched chain amino acids), and/or Urea Nitrogen); wherein a significant deviation of one or more measurements relative to the same measurements from a nonmetastatic control (such as, for example, a lower pH, higher PO₂, higher lactate, lower glucose, lower potassium, lower sodium, lower glutamine, and/or higher urea nitrogen) indicates the presence of LM.

The biological sample can be any tissue sample or liquid biopsy and can include, but is not limited to blood, plasma, and cerebrospinal fluid (CSF).

Also disclosed herein are methods of diagnosing LM, wherein the one or more blood gas, blood chemistry, mass spectrometry, or hematological measurements comprise a measurement of one or any combination of or more of 2, 3, 4, 5, 6, 7, 8 9, 10, 11, 12, 13, 14, 15, 16 17, 18, 19, 20, or more of chloride (Cl), sodium (Na), potassium (K), total CO₂ (TCO₂), Anion Gap, Ionized Calcium (iCa), Lactate, Glucose (Glu), Urea Nitrogen (BUN)/Urea, 3-methyl/4-methyl-2-oxovaleric acid, 3-methyl-2-oxobutanoic acid, Creatinine, Hematocrit, Hemoglobin, pH, partial pressure of carbon dioxide (PCO₂), partial pressure of oxygen (PO₂), bicarbonate (HCO₃), Base Excess, and/or sO₂. For example, the measurement can comprise Na and K; Na and glutamine; Na and lactate; K and glutamine; K and lactate; glutamine and lactate; Na, K, and glutamine; Na, K, and lactate; K, glutamine, and lactate; Na, K, glutamine, and lactate; SO₂ and K; SO₂ and Na; or NA, K, and sO₂. In one aspect, keto acid and/or amino acid levels can be measured by mass spectrometry.

In one aspect, disclosed herein are methods of diagnosing LM, wherein the one or more blood gas, blood chemistry, mass spectrometry, or hematological measurements are obtained using a point-of-care body fluid analyzer (such as, for example an iSTAT). In some aspect, the one or more blood gas, blood chemistry, or hematological measurements are on a cartridge (such as, for example an iSTAT CHEM8 or CG4 cartridge) for use with a body fluid analyzer. In one aspect, keto acid and/or amino acid levels can be measured by mass spectrometry.

The disclosed methods are not limited to metastatic cancers, but can include primary lymphomas which occur in the CSF and expand to the meninges.

In one aspect, the diagnostic method can further comprise performing cytology, proteomics, metabolomics, and/or or lipidomics on the sample.

### C. Method of Treating Cancer

As noted above, diagnosis of LM can be challenging. Moreover, as the present standard of care involve intrathecal administration of a therapeutic which is highly invasive. Due to the dangers of intrathecal administration, physicians are hesitant to initiate treatment unless diagnosis is definitive.

Disclosed herein are methods of treating, inhibiting, reducing, decreasing, ameliorating, and/or preventing LM of a cancer (such as, for example, melanoma, breast cancer, lung cancer, leukemia, or lymphoma) in a subject comprising: a) obtaining a biological sample (such as, for example, a tissue sample, or liquid biopsy including, but not limited to blood, plasma, or cerebrospinal fluid (CSF)); b) obtaining one or more blood gas, blood chemistry, mass spectrometry, or hematological measurements (such as, for example, a measurement of pH, PO₂, Lactate, Glucose, Sodium, Potassium, Glutamine, keto acid levels (including, but not limited to branched chain keto acids (such as, for example, 3-methyl/4-methyl-2-oxovaleric acid and 3-methyl-2-oxobutanoic acid)), amino acid levels (including, but not limited to branched chain amino acids), and/or Urea Nitrogen) in the biological sample; wherein a significant deviation of one or more measurements relative to the same measurements from a nonmetastatic control (such as, for example, a lower pH, higher PO₂, higher lactate, lower potassium, lower sodium, lower glutamine, lower glucose, and/or higher urea nitrogen) indicates the presence of LM; and c) administering to the subject anti-cancer therapy (such as, for example, radiation therapy, intrathecal chemotherapy, or immunotherapy). In one aspect, the diagnostic method can further comprise performing cytology, proteomics, metabolomics, and/or or lipidomics on the sample.

In one aspect, disclosed herein are methods of treating, inhibiting, reducing, decreasing, ameliorating, and/or preventing LM, wherein the one or more blood gas, blood chemistry, mass spectrometry, or hematological measurements comprise a measurement of one or any combination of or more of 2, 3, 4, 5, 6, 7, 8 9, 10, 11, 12, 13, 14, 15, 16 17, 18, 19, 20, or more of chloride (Cl), total CO₂ (TCO₂), Anion Gap, Ionized Calcium (iCa), Lactate, Sodium (Na), Potassium (K), Glutamine, Glucose (Glu), 3-methyl/4-methyl-2-oxovaleric acid, 3-methyl-2-oxobutanoic acid, Urea Nitrogen (BUN)/Urea, Creatinine, Hematocrit, Hemoglobin, pH, partial pressure of carbon dioxide (PCO₂), partial pressure of oxygen (PO₂), bicarbonate (HCO₃), Base Excess, and/or sO₂. For example, the measurement can comprise Na and K; Na and glutamine; Na and lactate; K and glutamine; K and lactate; glutamine and lactate; Na, K, and glutamine; Na, K, and lactate; K, glutamine, and lactate; Na, K, glutamine, and lactate; SO₂ and K; SO₂ and Na; or NA, K, and sO₂.

Also disclosed herein are methods of treating, inhibiting, reducing, decreasing, ameliorating, and/or preventing LM, wherein the one or more blood gas, blood chemistry, mass spectrometry, or hematological measurements are obtained using a point-of-care body fluid analyzer (such as, for example an iSTAT). In some aspect, the one or more blood gas, blood chemistry, or hematological measurements are on a cartridge (such as, for example an iSTAT CHEM8 or CG4 cartridge) for use with a body fluid analyzer. In one aspect, keto acid and/or amino acid levels can be measured by mass spectrometry.

In one aspect, when LM is diagnosed, the patient can receive radiation therapy, intrathecal chemotherapy, anti-cancer agents, and/or immunotherapy (such as, for example, immune checkpoint inhibitors, and/or adoptive cell transfer of chimeric antigen receptor (CAR) T cells, CAR Natural Killer (NK) cells, CAR macrophage (CARMA), tumor infiltrating lymphocytes (TILs), marrow infiltrating lymphocytes (MILs)) or other anti-cancer treatment.

The disclosed compositions can be used to treat LM developing from any disease where uncontrolled cellular proliferation occurs such as cancers. A representative but non-limiting list of cancers giving rise to LM that the disclosed methods can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

It is understood that once treatment of LM has been administered, or treatment of a caner that can give rise to LM has commenced, the disclosed methods can also be used to monitor the efficacy of the therapeutic regimen. Accordingly, in one aspect, disclosed herein are methods of monitoring the efficacy of a therapeutic regimen for treating leptomeningeal metastasis (LM) of a cancer (such as, for example, melanoma, breast cancer, lung cancer, leukemia, or lymphoma) in a subject comprising: a) obtaining a cerebrospinal fluid (CSF) sample; and b) obtaining one or more blood gas, blood chemistry, or hematological measurements in the CSF (such as, for example, a measurement of pH, PO₂, Lactate, Glucose, Sodium, Potassium, Glutamine, and/or Urea Nitrogen); wherein a significant deviation of one or more measurements relative to the same measurements from a nonmetastatic control (such as, for example, a lower pH, higher PO₂, higher lactate, lower glucose, lower potassium, lower sodium, lower glutamine, keto acid levels (including, but not limited to branched chain keto acids (such as, for example, 3-methyl/4-methyl-2-oxovaleric acid and 3-methyl-2-oxobutanoic acid)), amino acid levels (including, but not limited to branched chain amino acids), and/or higher urea nitrogen) indicates the therapeutic regiment is not efficacious. Similarly, a lack of significant deviation and/or a higher pH, lower PO₂, lower lactate, higher glucose, higher potassium, higher sodium, higher glutamine, and/or lower urea nitrogen indicates the treatment regimen is efficacious.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Cloforex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Leptomeningeal Metastasis Diagnostics

Leptomeningeal metastasis (LM) is a terminal cancer condition that occurs when tumor cells spread to cerebrospinal fluid and the meninges. LM of melanoma (LMM) is an aggressive terminal complication for cancer patients. Left untreated survival is 6-8 weeks. Estimates of the frequency of LMM range from 0.8% to 8%; however, these estimates are based on current unreliable diagnostics methods and the frequency could be as high as 20%.

The most common malignancies that metastasize to the leptomeninges are breast cancer (12-35%), lung cancer (10-26%), melanoma (5-25%), leukemia and lymphoma. In this talk I will specifically focus on leptomeningeal melanoma metastasis. Typically, patients present with symptoms of CNS dysfunction including hydrocephalus, seizures, cognitive changes, headache, gait difficulty, and pain in the neck or back.

Currently standard of care treatment protocols for leptomeningeal carcinomatosis include radiation therapy (RT) and intrathecal chemotherapy (for example IL-2 administration) and present many challenges. Treatment of the entire neuroaxis with RT can result in significant adverse events for the patient, therefore RT of symptomatic sites coupled with chemotherapy is a more favorable approach. One recent study reported intrathecal chemotherapy to improve survival and symptom control in patients with LMM from non-small cell lung cancer, leading to median overall survival of 3 months—which is still quite dismal. Similarly, there has been a study of intrathecal IL-2 for these patients but proved to show low response and high toxicity.

Promisingly, there are some sparse individual case reports of systemic treatments with vemurafenib or checkpoint blockade with promising results in individual patients and at the moment there is a trial planned for testing systemic and intrathecal anti-PD1 therapy. While treatment outcomes for leukemia and lymphoma are relatively exceptional (methotrexate), most patients with leptomeningeal metastasis from solid tumors (especially lung and melanoma) deteriorate rapidly regardless of treatment

Some of the challenges of studying leptomeningeal carcinomatosis include how little we really know about the biology of cells homing to the leptomeninges. It is very difficult to study, especially in melanoma, due to the small sample size, both in terms of the number of patients who have clinically diagnosed LMM and the number of cells we can isolate from the patients. The biggest problem is that there are no effective therapies for most LMM patients.

Presently, the “gold standard” of diagnosis remains a positive CSF cytology which involves morphologic appearance of fixed cells and is therefore a somewhat primitive test with a very low sensitivity and positive predictive value. A single CSF cytology test results in a false negative result ˜50% of the time. Due to the diffuse nature of the tumor, imaging is very challenging.

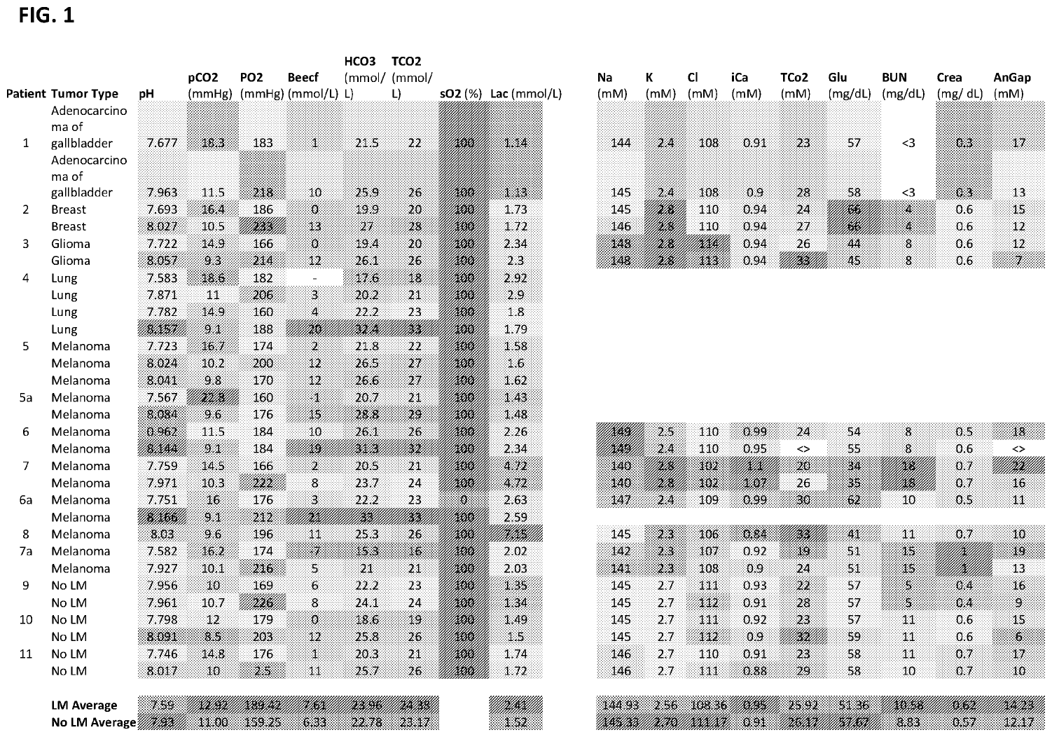

To develop a better LM diagnostic, a single point of care blood analyzer (iSTAT from Abbott) was used. iSTAT is designed to make blood gas, blood chemistry, or hematological measurements. This is accomplished through the use of cartridges which comprise particular tests. Here we used the CHEM8 cartridge to measure Na, K, Cl, total CO₂ (TCO₂), Anion Gap, Ionized Calcium (iCa), Lactate, Glucose (Glu), Urea Nitrogen (BUN)/Urea, Creatinine, Hematocrit, Hemoglobin; and the CG4 cartridge to measure take blood gas measurements such as pH, partial pressure of carbon dioxide (PCO₂), partial pressure of oxygen (PO₂), bicarbonate (HCO₃), Base Excess, and/or sO₂. However, rather than blood, CSF was obtain and tested. We found that pH, pO₂, lactate, glucose and urea nitrogen had significant deviations from non-LM patients (FIG. 1). In particular, the CSF of LM patients had lower pH, increased pO₂, increased lactate, lower glucose, and increased urea nitrogen.

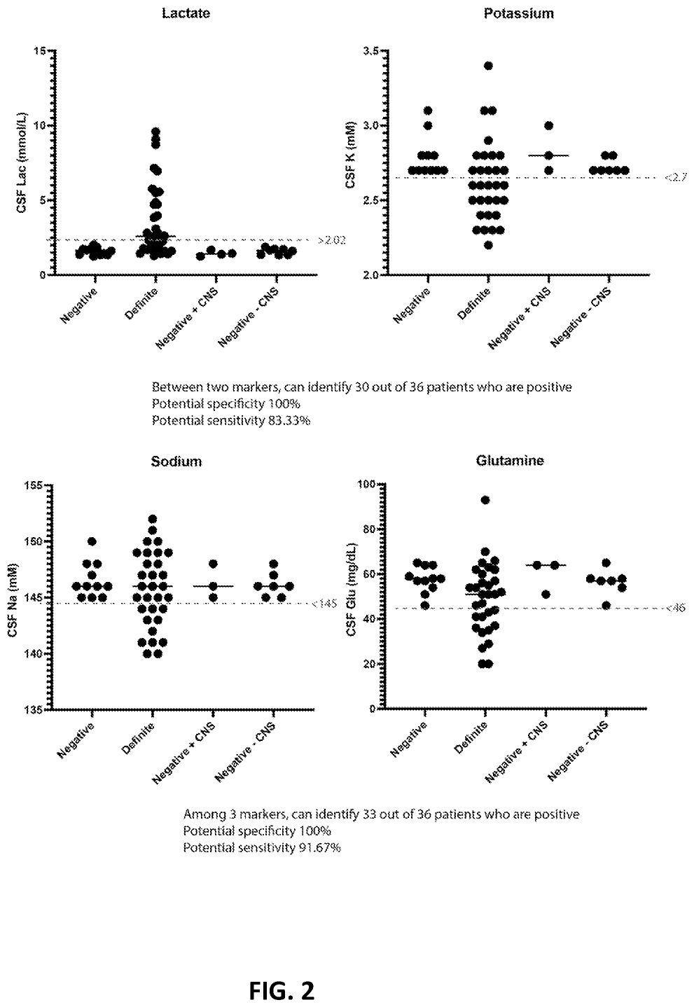

We further observed that LM patients had lower potassium and sodium relative to negative controls (FIG. 2). CSF was analyzed using iSTAT using CG4+ and Chem8 (Potassium, Sodium and Glutamine) cartridges. Patients with following tumor types included: Melanoma, Lung cancer, Breast Cancer, lymphoma, leukemia, glioma, pancreatic cancer and carcinoid tumor of stomach. Using increase in lactate and decrease in potassium alone identified 30 out of 36 patients that were later confirmed to be positive by MRI or cytology (sensitivity of 83.33%). The identification sensitivity improved to 91.67% (i.e., 33 out of 36) by also looking for a decrease in sodium or glutamine. No false positives were identified.

As shown in Table 1, we recently analyzed a new cohort of CSF samples from patients with LMD diagnosis, but these were stored samples and not freshly measured as previously (analyzed after freeze-thaw).

TABLE 1

| | Lac (mmol/L) | Na (mM) | K (mM) |
|---|---|---|---|
| LMD1 | 3.1 | 149 | 2.7 |
| LMD2 | 1.5 | 144 | 2.8 |
| LMD3 | 1.79 | 151 | . |
| LMD4 | 2.55 | 144 | 2.3 |
| LMD5 | 1.44 | 146 | 2.7 |
| LMD6 | 1.38 | 149 | 2.2 |
| LMD7 | 2.01 | 149 | 2.8 |

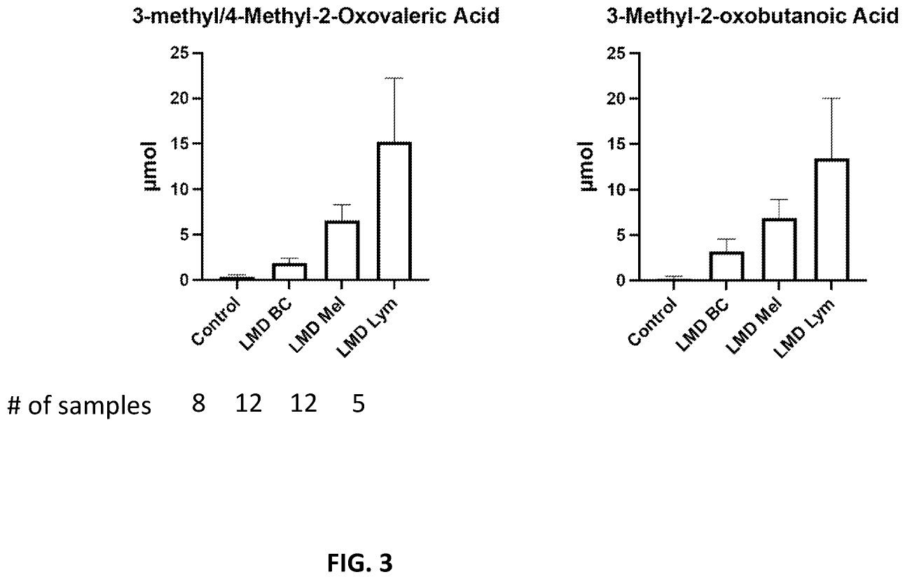

We have quantified absolute levels of BCKAs using mass spectrometry in CSF of cancer patients with no CNS involvement (control) or with LMD diagnosis from breast cancer (BC), melanoma (mel), and lymphoma (lym) (FIG. 3).

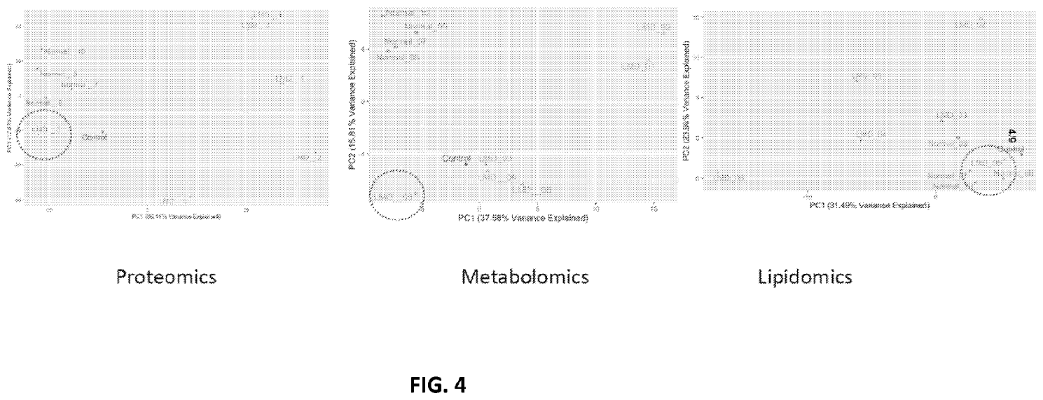

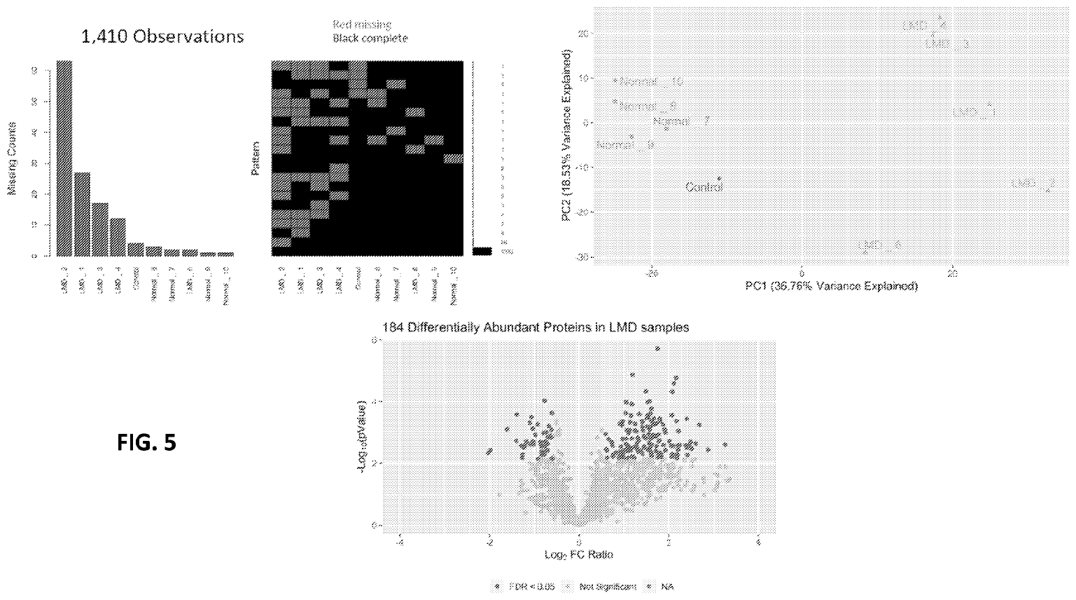

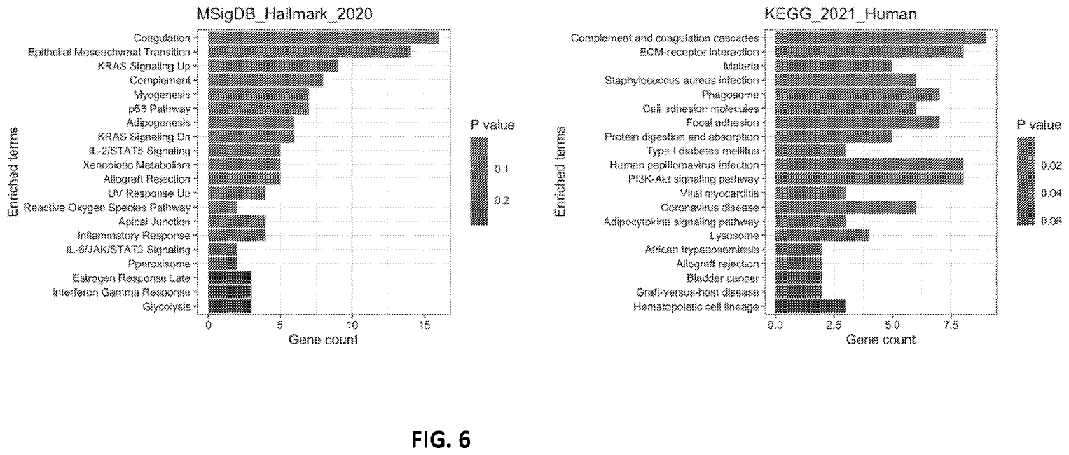

We also assayed the proteomics, metabolomic and lipidomics to see if there were observable differences between LMD patients and controls (FIG. 4). As shown in FIGS. 5 and 6, there were 184 differentially abundant proteins (FCN3, ICAM3, TDRD5, IGHA1 IGHA2, APOM, HP HPR, LPA, IGHM, C2, SERPINA3, FCN2, SERPINC1, IGLV1-47, ORM1, SAA4, PON1, MYH11, HLA-A, DEFA1 DEFA1B DEFA3, GP1BA, AZGP1, APOC1, IGHG2, HABP2, IGLC2 IGLC3 IGLC6, CPN1, F11, IGKC, IGFALS, CPB2, F9, IGHG1, LILRA1 LILRA3 LILRB1, C8B, ITIH3, PPIA PPIAL4A PPIAL4C PPIAL4D PPIAL4E PPIAL4F PPIAL4G PPIAL4H, XPNPEP3, ADIPOQ, LRG1, HLA-C, RSL1D1, FUCA1, GUCA2A, GPNMB, IGKV2-28 IGKV2-30 IGKV2-40 IGKV2D-28 IGKV2D-29 IGKV2D-30 IGKV2-29 IGKV2D-26, IGKV3-15 IGKV3-7 IGKV3D-7, PTMS, KRT6B, IGLL5 IGLC1 IGLV2-11 IGLV2-8, DPP10, GASK1B, ICAM1, IGKV3D-11, KPRP, C5, EVC2, SECTM1, IGKV3-20, PPBP, SFN, LY6D, TECTA, PEPD, COTL1, MBL2, IGKV3D-20, ELFN2, CALD1, KLK8, IGHV3-15 IGHV3-72 IGHV3-73, PARK7, CDSN, PRDX1 PRDX4, DCD, IGHG3, TNFRSF1B, COL21A1, KIF13B, MSN RDX, A2M PZP, CD33, IGLV8-61, GPX3, VNN1 VNN3, YWHAG, LCP1, SH3BGRL, TAGLN, BST1, LHPP, COL6A2, BCAM, COMP, DPP4, C1RL, DSG1, SCGB1A1, DYNC1LI2, CFD, ANPEP, HSPA6 HSPA1L HSPA7, CSTA, FLNA, APOL1, CD93, GPR180, CST6, GLDN, FAH, ATRN, CAT, PDIA4, DSC2, TMEM132D, SULF1, THBS1, DDAH2, TMEM132C, IGHV1-3 IGHV1-69 IGHV1-69D IGHV1-8, PRSS8, LYPD3, TMEM132B, CNGA4, PCSK6, GLOD4, HTRA1, PAPLN, CD58, TBCA, NGFR, SLC2A13, PRH1 PRH2, EMILIN2, GRN, RNASE2, SPINT1, PCMT1, MXRA8, PLXDC1, COL4A2, IGLV3-10 IGLV3-16 IGLV3-25, GLV3-27, IFI27L2, HBEGF, EFEMP1, MSANTD3-TMEFF1 TMEFF1, ITM2B, PRRT3, TNFRSF21, SLC6A1, CNDP1, GPR158, CPE, GALNS, TNR, PAM, THBS2, CSPG5, ARSA, VWC2, NPY, SHISA7, LUZP2, EFEMP2, EFCAB14, SCN3B, VGF, SLIT2, PNOC, SCG2, NPTXR, SCG3, LINGO1, CDH4, CTSD, TAC3, CD81, FRAS1, GRIA4, TAC1, MMP24, WFDC1, FBLL1, and/or CCDC189) and these proteins were involved in coagulation, KRAS signalling up, complement, myogenesis, the p53 pathway, epithelial mesenchymal transition PI3K-Akt signaling pathway amongst other pathways.

### 2. Example 2: Untargeted Metabolomics

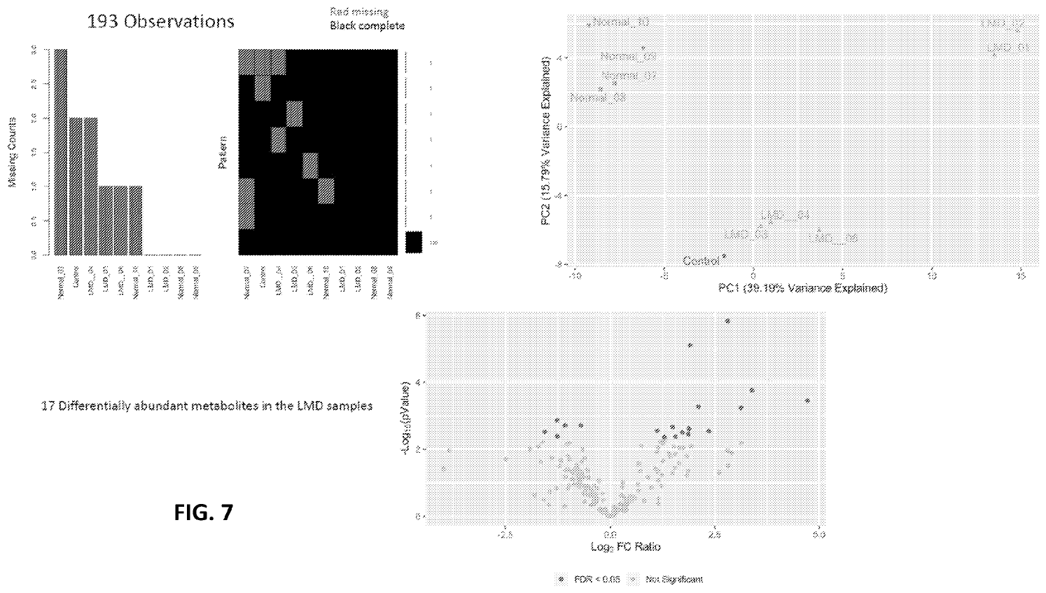

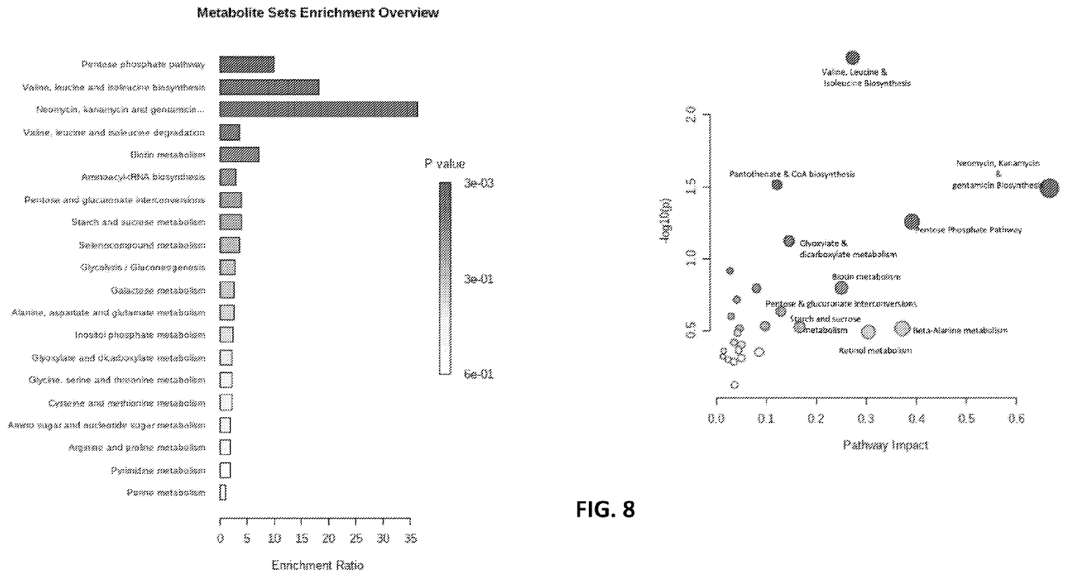

Cerebral spinal fluid (CSF) sample (100 μL) was spiked with 5 μL of a mixture of stable isotope-labeled standards (Cambridge Isotope Labs, Tewksbury, MA). An aliquot of pre-chilled methanol was added to each sample for a final composition of 80% methanol to extract metabolites and precipitate proteins. The samples were vortexed and incubated at −80° C. for 30 minutes. The protein was pelleted by centrifugation at 18,000×g for 10 minutes at 4° C. The supernatant containing the metabolites was lyophilized and then re-suspended in 100 μL of 80% methanol. A 10 μL aliquot of each sample was used to make a pool sample. As shown in FIGS. 7 and 8, LMD patients differed in 17 abundant metabolites (3-Methyl-2-Oxovaleric acid; 4-Methyl-2-Oxovaleric acid, O-Acetyl-L-carnitine, Glyoxylic acid, Indoxyl sulfate, Octanoyl-L-Carnitine, Decanoyl-L-Carnitine, Butyryl-L-Carnitine, Octanoyl-L-Carnitine, 3-Aminoisobutanoate; 2-Amino-2-Methylpropanoate, Deoxycytidine, L-Methionine, L-Methionine, L-Alanine; D-Alanine; Beta-Alanine, Glutaryl-L-Carnitine, 3-Hydroxy-3-methylglutarate, D-Ribose 5-phosphate; Xylulose 5-phosphate, Biotin, D-Glucose-6-phosphate, and/or Retinoate) and the enriched metabolite sets segregated into the pentose phosphate pathway; the valine, leucine, and isoleucine biosynthesis pathway, and the neomycin, kanamycin, and gentamicin biosynthesis pathway.

Liquid chromatography-high resolution mass spectrometry (LC-HRMS) was performed using a UHPLC (Vanquish, Thermo Scientific) interfaced with a hybrid quadrupole-Orbitrap mass spectrometer (Q Exactive HF, Thermo Scientific, San Jose, CA). An aliquot (5 μL) of each sample was loaded onto a SeQuant ZIC-pHILIC guard column (4.6 mm inner diameter/ID×20 mm length, 5 μm particle size, Millipore Sigma, Burlington, MA) connected to a SeQuant ZIC-pHILIC column (4.6 mm ID×150 mm length, 5 μm particle size, MilliporeSigma, Burlington, MA) maintained at 30° C. The following solvent system was used for LC-MS analysis: solvent A was aqueous 10 mM ammonium carbonate and 0.05% ammonium hydroxide and solvent B was 100% acetonitrile. A linear gradient was programmed from 80 to 20% B over 13 minutes with a flow rate of 0.250 mL/min, and then maintained at 20% B for 2 minutes, followed by re-equilibration over 5 minutes at a flow rate of 0.250 m/min, for a total run time of 20 minutes for each experiment. The Q Exactive HF mass spectrometer was operated in positive and negative mode separately using a scan range from m/z 60 to m/z 900. LC-MS data files were converted to mzXML files using ProteoWizard and analyzed using MZmine 2.38.

### 3. Example 3: Untargeted Lipidomics

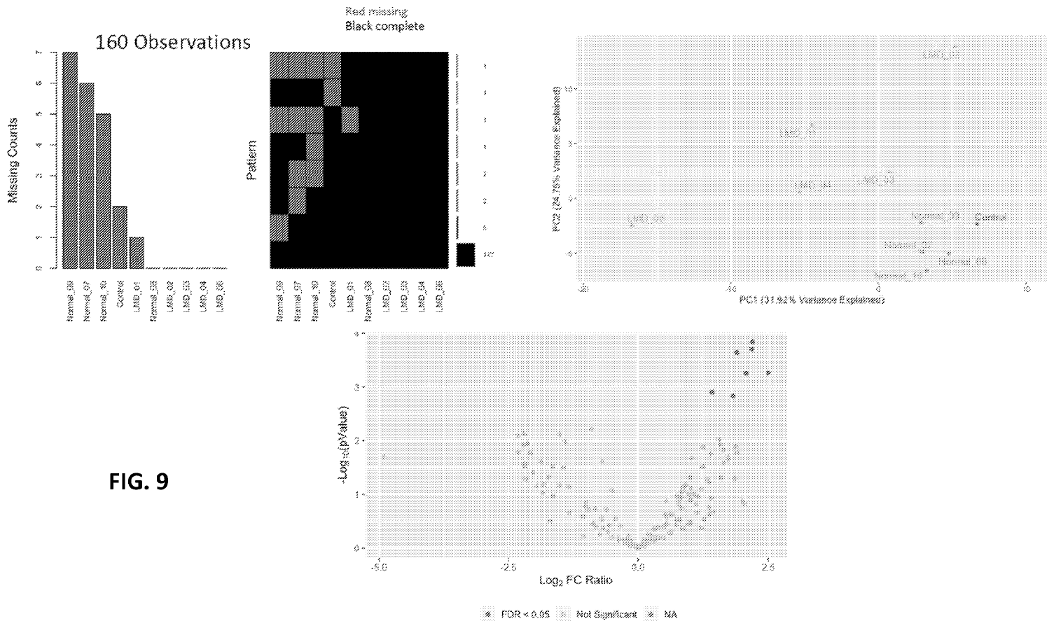

A 200 μL aliquot of each CSF sample was spiked with 5 μL of SPLASH Lipidomix standard (Avanti Polar Lipids) and extracted with 600 μL of pre-chilled isopropanol. The samples were vortexed and incubated at −80° C. for 20 minutes. The protein was pelleted by centrifugation at 13,800×g for 20 minutes at 4° C., and its concentration was calculated using Bradford assays to estimate total protein content (Pierce™ Coomassie (Bradford) Protein Assay Kit, 23200, Thermo Scientific). The supernatant containing the lipids was lyophilized and then re-suspended in 100 μL of 100% methanol. As shown in FIG. 9, lipidomics showed 7 differentially abundant lipids (C28 H48 O7 N1 P1, C44 H80 O7 N1 P1, C44 H82 O7 N1 P1, C46 H84 O7 N1 P1, C30 H52 O7 N1 P1, C59 H90 O4, and/or C30 H52 O7 N1 P1) between LMD patients and normal controls.

The analysis was performed using LC-MS/MS with a Vanquish LC (Thermo, San Jose, CA) interfaced with a Q Exactive HF mass spectrometer (Thermo, San Jose, CA). Chromatographic separation was conducted on Brownlee SPP C18 column (2.1 mm×75 mm, 2.7 μm particle size, Perkin Elmer, Waltham, MA) using mobile phase A containing 100% water with 0.1% formic acid and 1% of 1M ammonium acetate and mobile phase B containing 1:1 acetonitrile:isopropanol with 0.1% formic acid and 1% of 1M ammonium acetate. The gradient was programmed as follows: 0-2 min 35% B, 2-8 min from 35% B to 80% B, 8-22 min from 80% B to 99% B, 22-36 min 99% B, 36.1-40 min from 99% to 35% B at a flow rate of 0.400 m/min. Full MS and tandem mass spectrometry using data dependent acquisition (top-10 method) was used in both positive and negative mode separately. Lipid data was analyzed using LipidSearch 4.2 (Thermo Fisher Scientific) and normalized by the amount of protein in the sample.

1. A method of diagnosing leptomeningeal metastasis (LM) of a cancer in a subject comprising:
a) obtaining a biological sample; and
b) obtaining one or more blood gas, blood chemistry, or hematological measurements in the biological sample;
wherein a significant deviation of one or more measurements relative to the same measurements from a nonmetastatic control indicates the presence of LM.

2. The method of diagnosing LM of claim 1, wherein the biological sample comprises cerebrospinal fluid (CSF), blood, or plasma.

3. The method of diagnosing LM of claim 1, wherein the one or more blood gas, blood chemistry, mass spectrometry, or hematological measurements comprise a measurement of one or any combination of two or more of chloride (Cl), total CO2 (TCO2), Anion Gap, Ionized Calcium (iCa), Lactate, Glucose (Glu), Sodium, Potassium, Glutamine, keto acid levels, amino acid levels, Urea Nitrogen (BUN)/Urea, Creatinine, Hematocrit, Hemoglobin, pH, partial pressure of carbon dioxide (PCO2), partial pressure of oxygen (PO2), bicarbonate (HCO3), Base Excess, and/or sO2.

4. (canceled)

5. The method of diagnosing LM of claim 3, wherein a lower pH, higher PO2, higher lactate, lower glucose, lower potassium, lower sodium, lower glutamine, and/or higher urea nitrogen indicates LM.

6. The method of diagnosing LM of claim 3, wherein the keto acid levels measured are 3-methyl/4-methyl-2-oxovaleric acid and 3-methyl-2-oxobutanoic acid.

7. The method of diagnosing LM of claim 1, wherein the one or more blood gas, blood chemistry, or hematological measurements are obtained using a point-of-care body fluid analyzer.

8. The method of diagnosing LM of claim 7, wherein the one or more blood gas, blood chemistry, or hematological measurements are on a cartridge for use with a body fluid analyzer.

9. The method of diagnosing LM of claim 7, wherein the point of care body fluid analyzer is an iSTAT.

10. The method of diagnosing LM of claim 7, wherein the one or more blood gas, blood chemistry, or hematological measurements are on a CHEM8 or CG4 cartridge.

11. The method of diagnosing LM of claim 1, wherein the cancer comprises melanoma, breast cancer, lung cancer, leukemia, or lymphoma.

12. A method of treating LM in a subject comprising:
a) obtaining a biological sample;
b) obtaining one or more blood gas, blood chemistry, or hematological measurements in the biological sample; wherein a significant deviation of one or more measurements relative to the same measurements from a nonmetastatic control indicates the presence of LM; and
c) administering to the subject anti-cancer therapy.

13. The method of diagnosing LM of claim 12, wherein the biological sample comprises cerebrospinal fluid (CSF), blood, or plasma.

14. The method of treating LM of claim 12, wherein the one or more blood gas, blood chemistry, or hematological measurements comprise a measurement of one or more of Chloride (Cl), total CO2 (TCO2), Anion Gap, Ionized Calcium (iCa), Lactate, Sodium (Na), Potassium (K), Glutamine, Glucose (Glu), Urea Nitrogen (BUN)/Urea, keto acid levels, amino acid levels, Creatinine, Hematocrit, Hemoglobin, pH, partial pressure of carbon dioxide (PCO2), partial pressure of oxygen (PO2), bicarbonate (HCO3), Base Excess, and/or sO2.

15. (canceled)

16. The method of treating LM of claim 14, wherein the one or more blood gas, blood chemistry, or hematological measurements are obtained using a point-of-care body fluid analyzer.

17. The method of treating LM of claim 16, wherein the one or more blood gas, blood chemistry, or hematological measurements are on a cartridge for use with a body fluid analyzer.

18. The method of treating LM of claim 16, wherein the point of care body fluid analyzer is an iSTAT.

19. The method of treating LM of claim 18, wherein the one or more blood gas, blood chemistry, or hematological measurements are on a CHEM8 or CG4 cartridge.

20. The method of diagnosing LM of claim 14, wherein the keto acid levels measured are 3-methyl/4-methyl-2-oxovaleric acid and 3-methyl-2-oxobutanoic acid.

21. The method of treating LM of claim 12, wherein the cancer comprises melanoma, breast cancer, lung cancer, leukemia, or lymphoma.

22. The method of treating LM of claim 12, wherein the anti-cancer therapy comprises radiation therapy, intrathecal chemotherapy, or immunotherapy.
